(19) United States
(12) Patent Application Publication
(10) Pub. No.: US 2020/0249172 A1
(43) Pub. Date: Aug. 6, 2020

(54) Colorimetric Detection of Organic Amines Using Metal-Organic Frameworks

(71) Applicant: The Johns Hopkins University, Baltimore, MD (US)

(72) Inventors: James K. Johnson, Silver Spring, MD (US); Kelly A. Van Houten, West Friendship, MD (US)

(21) Appl. No.: 16/556,397

(22) Filed: Aug. 30, 2019

Related U.S. Application Data
(60) Provisional application No. 62/800,635, filed on Feb. 4, 2019.

(51) Int. Cl. G01N 21/78; G01N 33/52; G01N 33/94

(52) U.S. Cl. CPC G01N 21/78; G01N 2035/00188; G01N 33/9486; G01N 33/52

(57) ABSTRACT

Provided is an article for detecting organic amines, wherein the article includes a solid support impregnated with an indicator reagent comprising a metal-organic framework structure. Also provided is a method of detecting organic amines, wherein the method includes the step of exposing an article comprising a solid support impregnated with an indicator reagent to a medium including an organic amine to produce a color change, wherein the indicator reagent includes a metal-organic framework structure.

### CROSS-REFERENCE TO RELATED APPLICATIONS

This application claims the benefit of U.S. Provisional Patent Application No. 62/800,635 filed on Feb. 4, 2019, the entire contents of which are hereby incorporated herein by reference.

### BACKGROUND

Embodiments of the invention described herein generally relate to a method, composition, article, and device for the detection of chemicals. More specifically, embodiments of the invention relate to a method, composition, article, and device for colorimetric detection of organic amines using all indicator reagent containing metal-organic frameworks.

According to data provided by the National Institute of Drug Abuse, more than 130 people in the United States die every day after overdosing on opioids. The misuse of and addiction to opioids—including prescription pain relievers, heroin, and synthetic opioids such as fentanyl—is a serious national crisis that affects public health as well as social and economic welfare. The increase in drug use has also contributed to the spread of infectious diseases including HIV and hepatitis C. The Centers for Disease Control and Prevention estimates that the total “economic burden” of prescription opioid misuse alone in the United States is 578,5 billion a year, including the costs of healthcare, lost productivity, addiction treatment, and criminal justice involvement.

Fentanyl is one of the most potent opioid substances used as a narcotic analgesic supplement in general and regional anesthesia as well as in management of persistent, severe chronic pain. Alarming epidemiological and forensic medicine reports, accumulated mainly during the last two decades, point to a growing increase in illicit use of fentanyl. The drug is commonly used to adulterate heroin as well as counterfeit prescription pain pills and sedatives that are purchased on the street. Increasing numbers of overdose deaths among cocaine users are also reported to be related to fentanyl-adulterated cocaine.

Although some users seek out fentanyl, it is often ingested unintentionally. Because of fentanyl's extremely high potency (it is 50-100 times more potent than morphine) and its ability to readily cross the blood-brain barrier, it can be lethal to breathe air with aerosolized fentanyl in it or touch a contaminated surface. Detection of even minor amounts of fentanyl and its derivatives is therefore critical to avoid overdoses, poisoning, and ensure public safety.

Specific color chemistry methods are known to detect opioid drugs. However, these methods are generally not suitable for rapid field analysis. In one of them, a paper swipe containing the drug is sprayed by at least one reagent to develop a color. This method is inaccurate because the paper swipe may develop a color change itself, which must be ignored. In addition, all color changes due to application of the reagent may be dramatically affected by the precision of spraying and the ambient temperature of the test. Other colorimetric detection methods are similarly not suitable. For example, a Fen-Her™ test, which is a thin-layer chromatography test that allows to clearly differentiate fentanyl and heroin, takes about 13 minutes to develop. Antibody test strips are also unreliable, as they detect only metabolites, but not the parent compounds. In addition, some antibody test strips require a specific buffer and do not provide reliable detection of drugs in an aqueous solution.

### SUMMARY

An embodiment provides an article for detecting organic amines. The article includes a solid support impregnated with an indicator reagent, wherein the indicator reagent includes a metal-organic framework structure.

Another embodiment provides a device including the article for detecting organic amines.

Still another embodiment provides a composition including a solid support impregnated with an indicator reagent, wherein the indicator reagent includes a metal-organic framework structure.

Yet another embodiment provides a method of detecting organic amines. The method includes the step of exposing an article comprising a solid support impregnated with an indicator reagent to a medium including an organic amine to produce a color change of the indicator reagent, wherein the indicator reagent includes a metal-organic framework structure.

### BRIEF DESCRIPTION OF THE DRAWINGS

The present application contains at least one drawing executed in color. Copies of this patent application with color drawing(s) will be provided by the Office upon request and payment of the necessary fire.

The above and other aspects and features of the present disclosure will become more apparent by describing in detail exemplary embodiments thereof with reference to the attached drawings, in which:

FIG. 1 is a flow chart illustrating the steps of the method described herein;

FIG. 2 is a photograph showing the colorimetric response of the ZnSiF₆(DPNDI)₂⊃Naphthalene-impregnated filter paper treated with fentanyl;

FIG. 3 is a photograph showing the colorimetric response of the fentanyl-sensing metal-organic frameworks (FMOF) with respect to some drug substances; and

FIG. 4 is a photograph showing the colorimetric response of the fentanyl-sensing metal-organic frameworks (FMOF) with respect to some drug substances.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below by referring to the figures to explain aspects of the present disclosure. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third, etc., may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross-section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“Substantially” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “substantially” can mean within one or more standard deviations, or within ±30%, 20%, 10%, 5% of the stated value.

As used herein, the term “alkylene group” refers to a divalent group derived from a straight or branched chain saturated aliphatic hydrocarbon having the specified number of carbon atoms.

As used herein, the term “alkenylene group” refers to a divalent group derived from a straight or branched chain unsaturated aliphatic hydrocarbon including at least one double bond and having the specified number of carbon atoms.

As used herein, the term “alkynylene group” refers to a divalent group derived from a straight or branched chain unsaturated aliphatic hydrocarbon including at least one triple bond and having the specified number of carbon atoms.

As used herein, the term “cycloalkylene group” refers to a divalent group having one or more saturated rings in which all ring members are carbon and having the specified number of carbon atoms.

As used herein, the term “cycloalkenylene group” refers to a divalent group having one or more rings in which all ring members are carbon, having at least one double bond inside the one or more rings, and having the specified number of carbon atoms.

As used herein, the term “arylene” refers to a divalent group derived from an aromatic hydrocarbon containing at least one ring and having the specified number of carbon atoms. The term “arylene” may be construed as including a group with an aromatic ring fused to at least one cycloalkylene ring.

As used herein, the term “heteroarylene” refers to a divalent group derived from an aromatic hydrocarbon containing at least one ring that has atoms of at least two different elements as members of its ring(s), one of which is carbon, and having the specified number of carbon atoms.

As used herein, the term “substituted” means including at least one substituent such. as a halogen (F, Cl, Br, I), hydroxyl, amino, thiol, ketone, anhydride, sulfone, sulfoxide, sulfonamide, carboxyl, carboxylate, ester (including acrylates, methacrylates, and lactones), amide, nitrile, sulfide, disulfide, nitro, C₁₋₂₀ alkyl, C₃₋₂₀ cycloalkyl (including adamantyl), C₁₋₂₀ alkenyl (including norbornenyl), C₁₋₂₀ alkoxy, C₂₋₂₀ alkenoxy (including vinyl ether), C₆₋₃₀ aryl, C₆₋₃₀ atyloxy, Co₇₋₃₀ alkylaryl, or C₇₋₃₀ alkylaryloxy.

Present embodiments of the invention provide an article for detecting organic amines. The article includes a solid support impregnated with an indicator reagent including a metal-organic framework structure. The indicator reagent produces a detectable change upon contact with an organic amine, thus providing a robust, reliable, and sensitive approach for rapidly detecting small amounts of opioid drugs.

The solid support may include a porous material having absorbing capacity. Such porous materials may be provided in a variety of physical forms. In an embodiment, the solid support may include a polymeric fiber, which may be produced from naturally occurring or synthetic polymer materials. Some polymeric fibers may be cellulose-based polymeric fibers, which may include naturally occurring cellulosic materials as well as synthetically-modified and/or regenerated cellulose materials. Synthetically-modified and/or regenerated cellulosic materials include cellulose and polysaccharide derivatives, including alkyl celluloses, hydroxyalkyl celluloses, cellulose ethers, cellulose esters, nitrocelluloses, and chitosan. Non-limiting examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose (CMC), cellulose triacetate, and cellulose sulfate sodium salt. Additional synthetic cellulosic materials may include, but are not limited to, rayon, rayon acetate, viscose rayon, and lyocell, Natural cellulosic materials that may be utilized in any one or more embodiments of the disclosure include, for example, cotton, linen, combinations, and derivatives thereof. Other materials that may be utilized in any one or more embodiments of the disclosure include, for example, bast fibers or other fibers derived from plant stems or barks such as, for example, flax, hemp, jute, ramie, and derivatives thereof. Other materials can include manmade cellulosic materials such as, for example, rayon, rayon acetate, viscose rayon, lyocell, and combinations thereof. Synthetically modified natural polymers of cellulose derivatives may be utilized in any one or more embodiments.

An example of a solid support is porous paper, such as filtering paper, which is

absorbent due to its excellent liquid distribution property. Another example of the solid support is cotton, the molecular structure of which offers a multitude of spaces that attract liquids and therefore provide excellent absorption. For example, a cotton swab may be conveniently used. The polymeric support may also be a fabric, which may be natural or synthetic. Non-limiting examples of the natural fabric include wool, mohair, and silk. Non-limiting examples of the synthetic polymeric fabric include polyaramids (e.g., liquid crystal polymers), polyamides (e.g., nylon), and polyesters (e.g., polyethylene terephthalate). Non-limiting examples of synthetic polymers include polyvinylidene fluoride, polymethylacrylate, hexadecyl acrylate. Nylon 11 (ω-undecanamide), Nylon 6, polyvinylchloride, and poly(2-hydroxyethylmethacrylate).

In other embodiments, the solid support may be a powder, for example, a crystalline powder or an amorphous powder. The solid support may be provided as a mobile solid form and may be used in association with “simulated moving bed” technology described in the art (Rodrigues A. E. et al. “Simulated Moving Bed Technology: Principles, Design, and Process Applications”, 2015, Butterworth-Heinemann, 1^{st} Edition, 288 pages) and incorporated herein in its entirety by reference.

The solid support may be impregnated with an indicator reagent including a metal-organic framework structure, which changes color upon its contact with an organic amine. As used herein, the term “metal organic framework” or “MOE” refers to crystalline compounds in which metal ions (or clusters of the metal ions) are coordinated to substantially rigid organic molecules. MOF of present embodiments of the invention form porous three-dimensional structures and may be represented by Formula 1:

[M]ₓ[LIG]_{y} Formula 1

In Formula I

M is a metal ion,

LIG is a polydentate ligand, and

x and y are positive numbers respectively defining relative stoichiometries of M and LIG in the MOF.

The MOF of present embodiments of the invention essentially include a metal-ligand complex, in which a polydentate ligand is coordinated to two or more metals. The metal may be any metal capable of coordinating to a polydentate ligand. In an embodiment, the metal may be a transition metal. As used herein, the term “transition metal” refers to an element whose atom has a partially filled d sub-shell, or which can give rise to cations with an incompleted sub-shell. As used herein, the term “polydentate ligand” refers to ligands comprising two or more atoms capable of binding a central metal ion in a coordination complex. The polydentate ligand is in contrast to monodentate ligands where only one atom can coordinate (e.g., as per molecules such as H₂O or EtOH). In an embodiment, the polydentate ligand may be a bidentate ligand that bind with two metals or metal ions. The bidentate ligands are also referred to as the chelating ligands.

Individual MOF may form MOF structures. In an embodiment, the MOF structure may include a complex in which a transition metal ion has a coordination number between 2 and 9, for example, 2, 3, 4, 5, 6, 7, 8, or 9, as discernable by crystallography. The coordination number may vary for each metal-ligand combination, and individual ligands may in some cases bridge between metal ions. The metal-ligand complex may be neutral, positively charged, or negatively charged.

The metal ions (M) and polydentate ligand (LIG) may constitute at least 50% of the molecular weight of the MOF structure. Though the metal ions (M) and polydentate ligand (LIG) constitute the majority of the molecular weight of the MOF structure, other species, such as solvates and/or auxiliary counterions (whether counterions to the metal ions or counterions to the ligand), may also be present in the complexes.

The MOF structure may be provided as a salt, For instance, when the metal-ligand complex is positively charged (e.g., if there are insufficient negatively charged polydentate ligand species within the complex to neutralize the positively charged metal ions), the MOF structure may be provided as a salt with an appropriate anion (e.g., a halide). Alternatively, where the metal-ligand complex is negatively charged (e.g., if the negative charges of the ligands outnumber the positive charges of the metal ions), the MOF structure may be provided as a salt with an appropriate cation (e.g., a metal ion, whether a transition metal or otherwise). In an embodiment, the MOF structure may be substantially neutral, in which the ligand charges balance with the metal ion charges.

In an embodiment, the respective oxidation (or ionization) states of M and LIG are of substantially equal magnitude but opposite polarity, As such, x and y may be substantially equal. and the metal-ligand complex may be substantially neutral.

The MOP structure may be provided as a solvate. In such circumstances, the solvate suitably includes solvent molecules coordinated directly to the metal ions (M). Alternatively, the MOF structure may be substantially free of solvate. The presence of solvates may affect the structure of the MOF, but will not generally affect the relative stoichiometry between the metal ions (M) and the polydentate ligands (LIG), assuming the solvate is uncharged. The MOF structure may include auxiliary counterions (e.g., halides, such as chloride). In such cases, the auxiliary cations may affect the relative stoichiometry between the metal ions (M) and the polydentate ligands (LIG). The new relative stoichiometry may depend on whether such auxiliary counterions are associated with the outer sphere of the complex or inner co-ordination sphere (i.e., directly coordinated with the meal ions). Auxiliary counterions may be those present in the original species used to form the MOF structures.

The relative stoichiometry of the metal ion (M) to polydentate ligand (LIG) depends on a number of factors, including the relative amounts of metal ions and polydentate ligand used to form the MOP structures, the oxidation state of the metal ions, the ionization state of the ligand, the presence of auxiliary counterions, and the presence of solvates. M and LIG are respectively present in the compound in a molar ratio of x:y. As such, the compound may be said to include species defined by the formula [M]ₓ[LIG]_{y}, where x and y respectively indicate the relative stoichiometries of M and LIG within the compound, both x and y being greater than zero.

In some embodiments, the MOF structures may he polydentate ligand-deficient, so that the co-ordination spheres of the metal ions are unsaturated with respect to the ligand. In other embodiments, the MOP structures may be oversaturated with polydentate ligand, for example, such that more ligands are involved in bridging between the metal ions. Still in other embodiments, the MOF structures may be somewhere between these two extremes. The proportions of metal ions to ligand may affect the structure and/or pore sizes of the MOF formed from the MOF structures.

The ratio of metal ion (M) to polydentate ligand (LIG) may be expressed as x:y or x/y. As mentioned above, this ratio, which reflects the relative stoichiometry between the metal ions (M) and polydentate ligand (LIG), may vary depending on a number of factors, and may in some embodiments be predetermined for optimum effect. For example, the ratio x:y may be between 10:1 and 1:10, between 5:1 and 1:5, between 2:1 and 1:2, or may he substantially 1:1. The x/y ratio may be such that the total charge of metal ions is at least 70% neutralized by the total charge of the polydentate ligand, at least 80% neutralized by the total charge of the polydentate ligand, at least 90% neutralized by the total charge of the polydentate ligand, or at least 95% neutralized by the total charge of the polydentate ligand. Any charge on the metal ions not neutralized by the polydentate ligand may be neutralized by auxiliary ligands or counterions. In an embodiment, the total charge of the metal ions may be substantially 100% neutralized by the charge of the polydentate ligand.

In some embodiments, the MOP structure of the invention may include a plurality of different ligands and/or auxiliary counterions. The MOF structure may include multiple different polydentate ligands, as defined herein. For instance, the LIG group may include multiple different LIG groups each independently defined as herein described in relation to the LIG group. For example, the LIG group may be represented as [LIG₁]_{y1}[LIG₂]_{y2} . . . [LIGₙ]_{yn} such that the MOP structure is defined by the Formula [M]ₓ[LIG₁]_{y1}[LIG₂]_{y2}. . . [LIGₙ]_{yn}.In such embodiments, y is the sum of the individual ligand stoichiometries, i.e., y=y1+y2+ . . . +yn. The MOP structure may include other auxiliary ligands, such as monodentate ligands and the like. In this manner, the MOF structure may be considered “doped”, so as to affect the structure and/or pore sizes to provide the optimal MOF for a given circumstance.

Likewise, the MOP structure of embodiments of the invention may also include a plurality of different metal ions, so long as at least one metal ion (M) is present. At least 10 weight % of the total metal ions present in the MOP-compound may be transition metal ions. For example, the transition metal may be included in an amount of at least 50 weight %, for example, at least 60 weight %, at least 70 weight %, at least 80 weight %, at least 90 weight %, or at least 95 weight %. In an embodiment, all of the metal ions present in the MOF structure may be transition metal ions.

M may be a transition metal ion, for example, from Groups 3 to 12 of the Periodic Table of elements. The transition metal ion is cationic, and may have an oxidation state of +1, +2, +3, or +4. In an embodiment, the transition metal ion may have the oxidation state of +2. For example, M may be Cu²⁺, Zn²⁺, Zr²⁺, Mn²⁺, and Co²⁺.

The polydentate ligand (LIG) is able to coordinate with M to provide a metal organic framework (MOF) structure. As such, LIG includes coordinating functionalities, that is, functional groups with a lone pair of electrons capable of coordinating with M.

In some embodiments, the LIG group may include a plurality of different LIG groups. For example, LIG may be represented as [LIG₁]_{y1}[LIG₂]_{y2} . . . [LIGₙ]_{yn}.

In an embodiment, LIG may be represented by Formula 2:

CORE—(—LINKER—R)ₙ Formula 2

In Formula 2,

n is an integer between 1 and 6 such that n individual and independently defined -LINKER-R groups (i.e., L₁-R₁ . . . Lₙ-Rₙ) are attached to CORE;

CORE includes one or more aromatic or heteroaromatic systems;

each LINKER group is the same or different, each being independently either absent (i.e., a single bond is connecting CORE with R) or a linking moiety selected from the group including C1-C10 alkylene, C2-C10 alkenylene, C2-C10 alkynylene, O, S, SO, SO₂, N(R′ₐ), CO, CH(OR′ₐ), CON(R′ₐ), N(R′ₐ)CO, N(R′ₐ)CON(R′ₐ), SO₂N(R′ₐ), N(R′ₐ)SO₂, OC(R′ₐ)₂, SC(R′ₐ) and N(R′ₐ)C(R′_{b})₂, wherein R′ₐ and R′_{b} are each independently hydrogen or C1-C8 alkyl;

each R group is the same or different, each being independently selected from a C6-C30 aryl or C1-C30 heteroaryl group bearing a lone pair of electrons capable of coordinating with M or substituted by a group bearing a lone pair of electrons capable of coordinating with M;

wherein CORE or any R group may be optionally substituted.

Any definitions herein regarding the LIG group may suitably encompass any acceptable ionized forms thereof. For example, references to LIG group substituents such as carboxylic acids may also include its conjugate base, that is a carboxylate anion.

The polydentate ligand suitably complexes directly with the transition metal ion (i.e., within the inner coordination sphere).

The polydentate ligand (LIG) may be a neutral species, for example, where auxiliary counterions serve to neutralize the charge of the metal ions. However, in some embodiments, LIG may include one or more ionized groups to thereby provide one or more anions. Such anions may then serve as counterions to the transition metal ions as well as coordinating groups within the metal-ligand complex. In an embodiment, LIG may include one or more ionized groups characterized as the conjugate base of an acid (e.g., carboxylate groups).

In certain embodiments, LIG includes either or both ionized and/or ionizable groups (e.g., carboxylate and carboxylic acid groups). The ratio of ionized to ionizable groups may be selectively varied, for example, by varying pH. The ratio of ionized to ionizable groups within LIG may be adapted so as to provide a substantially neutral metal-ligand complex. For example, LIG may include 3 coordinating groups, 2 of which are ionized (e.g., dicarboxylate) and 1 of which is a non-ionized ionizable group (e.g., carboxylic acid) so as to give an overall −2 charge capable of neutralizing a +2 charge on the metal ion.

For example, LIG may include 2 or more ionized and/or ionizable groups, for example, LIG may include 2 or more carboxylic and/or carboxylate groups.

The CORE group of the ligand may have n individual and independently defined -LINKER-R groups attached directly to appropriate positions) of the aromatic and/or heteroaromatic systems at different positions where n is greater than 1. The -LINKER-R groups may be juxtaposed around the CORE group to enable polydentate coordination with M, and optionally, also bridging between M units.

In an embodiment, the electron-deficient bidentate ligand may be a dicarboxylic acid ligand. For example, the dicarboxylic acid ligand may be represented by Formula 3:

HOOC—L¹—COOH Formula 3

In Formula 3, L¹ may be a single bond or a linking moiety including a substituted or unsubstituted C2-C30 alkenylene group, a substituted or unsubstituted C2-C30 alkynylene group, a substituted or unsubstituted C3-C30 cycloalkenylene group, a substituted or unsubstituted C3-C30 cycloalkynylene group, a substituted or unsubstituted C6-C30 arylene group, or a substituted or unsubstituted C6-C30 heteroarylene group, a C1-C30 alkylene group, wherein at least one non-adjacent —CH₂-group is replaced by —SO₂—, —C(═O)—, —O—, —S—, —SO—, —C(═O)O-, or C(═O)NR-, wherein R is hydrogen or a C1-C10 alkyl group, or a combination thereof

Non-limiting examples of the dicarboxylic acid ligand include the following compounds:

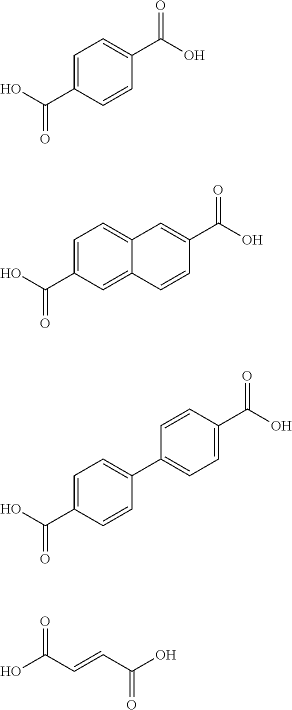 [SMILES: O=C(O)c1ccc(C(=O)O)cc1 ; O=C(O)c1ccc2cc(C(=O)O)ccc2c1 ; O=C(O)c1ccc(-c2ccc(C(=O)O)cc2)cc1 ; O=C(O)/C=C/C(=O)O]

In another embodiment, the electron-deficient bidentate ligand may be a heteroaromatic ligand including at least two nitrogen atoms. For example, the heteroaromatic ligand including at least two nitrogen atoms may be represented by Formula 4:

N-RING—L₂—N-RING Formula 4

In Formula 4,

N-RING may be the same or different and is selected from a nitrogen-containing heterocycle, and

L₂ is a single bond or a linking group.

The nitrogen-containing heterocycle may be a substituted or unsubstituted mono-heterocycle or a substituted or unsubstituted fused poly-heterocycle. The mono-heterocycle includes one ring having at least one nitrogen atom and may be a 5-membered heterocycle, a 6-membered heterocycle, or a 7-membered heterocycle, each of which may be saturated, partially saturated, or aromatic.

Non-limiting examples of the mono-heterocycles include pyrrole, 1-pyrroiine, 2-pyrroline, 3-pyrroline, pyrrolidine, imidazole, imidazoline, imidazolidine, pyrazole, 2-pyrazoline, 3-pyrazoline, pyrazolidine, 1,3,4,-triazole, 1,2,3-triazole, 1,2,4-triazole, tetrazole, pentazole, pyridine, pyridazine, pyrimidine, pyrazine, triazine, 1,4-dihydropyridine, dihydropyridazine, piperidine, piperazine, triazinane, azepine, azepane, 1H-1,2-diazepine, 1H-1,3-diazepine, 1H-1,4-diazepine, azocine, azocane, azonine, azonane, azecine, azecane, each of which may be substituted or unsubstituted.

Non-limiting examples of the fused poly-heterocycles include quinuclidine, 1,4-diazabicyclo[2.2.2]octane (DABCO), urotropine, indole, isoindole, 3H-indole, indoline, isoindoline, indolizine, 6-azaindole, 7-azaindole, benzimidazole, 1H-indazole, benzotriazole, 7-azabenzotriazole, purine, quinoline, isoquinoline, 4H-quinolizine, cinnoline, phthalazine, quinazoline, quinoxaline, 1,8-naphthyridine, ptcridine, carbazole, alpha-carboline, beta-carboline, gamma-carboline, delta-carboline, acridine, phenazine, o-phenaniroline, 1H-perimidine, 3-benzazepine, 1-benzazepine, 1H-1,4-benzodiazepine, 2,3,4,5-tetrahydro-1H-3-benzazepine, and dibenzazepine, each of which may be substituted or unsubstituted.

L² may be a single bond or a moiety including a substituted or unsubstituted C2-C30 alkenylene group, a substituted or unsubstituted C2-C30 alkynylene group, a substituted or unsubstituted C3-C30 cycloalkenylene group, a substituted or unsubstituted C3-C30 cycloalkynylene group, a substituted or unsubstituted C6-C30 arylene group, or a substituted or unsubstituted C6-C30 heteroarylene group, a C1-C30 alkylene group, wherein at least one non-adjacent —CH₂- group is replaced by —SO₂-, —C(═O)—, —O—, —S—, —SO—, —C(═O)O-, or —O(═O)NR-, wherein R is hydrogen or a C1-C10 alkyl group, or a combination thereof.

In an embodiment, the heteroaromatic ligand including at least two nitrogen atoms may be represented by Formula 5:

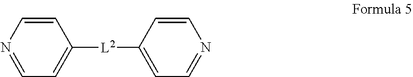

In Formula 5, L² may be a single bond or a moiety including a substituted or unsubstituted C2-C30 alkenylene group, a substituted or unsubstituted C2-C30 alkynylene group, a substituted or unsubstituted C3-C30 cycloalkenylene group, a substituted or unsubstituted C3-C30 cycloalkynylene group, a substituted or unsubstituted C6-C30 arylene group, or a substituted or unsubstituted C6-C30 heteroarylene group, a C1-C30 alkylene group, wherein at least one non-adjacent —CH₂- group is replaced by —SO₂—, —C(═O)—, —O—, —S—, —SO—, —C(═O)O-, or —C(═O)NR-, wherein R is hydrogen or a C1-C10 alkyl group, or a combination thereof.

Non-limiting examples of the heteroaromatic ligand including at least, two nitrogen atoms include the following compounds:

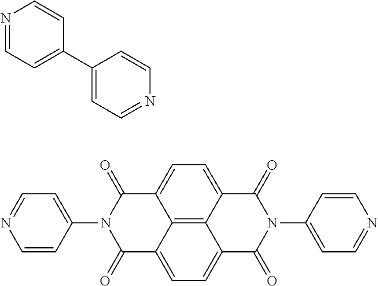 [SMILES: c1cc(-c2ccncc2)ccn1 ; O=C1c2ccc3c4c(ccc(c24)C(=O)N1c1ccncc1)C(=O)N(c1ccncc1)C3=O]

The MOF structure may further include an encapsulant. The encapsulant may be an electron rich molecule, which is capable of forming a donor-acceptor complex (i.e., a charge-transfer complex) by bonding to an electron-deficient polydentate ligand. In an embodiment, the encapsulant may be selected from a monocyclic aromatic compound, a bicyclic aromatic compound, a monocyclic heteroaromatic compound, or a bicyclic heteroaromatic compound. An example of the monocyclic aromatic compound may be a substituted or unsubstituted benzene. An example of the bicyclic aromatic compound may be a substituted or unsubstituted naphthalene. An example of the monocyclic heteroaromatic compound may be a substituted or unsubstituted pyridine, a substituted or unsubstituted pyridazine, a substituted or unsubstituted pyrimidine, a substituted or unsubstituted pyrazine, or a substituted or unsubstituted triazine. An example of the bicyclic heteroaromatic compound may be a substituted or unsubstituted quinoline, a substituted or unsubstituted isoquinoline, a substituted or unsubstituted cinnoline, a substituted or unsubstituted phthalazine, a substituted or unsubstituted quinazoline, or a substituted or unsubstituted quinaxoline.

The encapsulation may be carried out in a solvent, which may be an individual solvent or a mixture of two or more individual solvents. The solvent may dissolve and/or disperse the MOF components, and is not particularly limited as long as it does not chemically react with the MOF components. The solvent may be, for example, at least one selected from deionized water, methanol, ethanol, propanol, isopropanol, 2-methoxyethanol, 2-ethoxyethanol, 2-propoxyethanol, 2-butoxyethanol, methylcellosolve, ethylcellosolve, butyleellosolve, diethylene glycol methyl ether, diethylene glycol ethyl ether, dipropylene glycol methyl ether, toluene, xylene, hexane, heptane, octane, ethyl acetate, butyl acetate, diethylene glycol dimethyl ether, diethylene glycol dimethyl ethyl ether, methyl ethoxy propionate, ethyl ethoxy propionate, ethyl lactate, propylene glycol methyl ether acetate, propylene glycol methyl ether, propylene glycol propyl ether, methyl cellosolve acetate, ethyl cellosolve acetate, diethylene glycol methyl ether acetate, diethylene glycol ethyl ether acetate, acetone, methyl ethyl ketone, methyl isobutyl ketone, cyclopentanone, cyclohexanone, dimethyl formamide (DMF), N,N-dimethyl acetamide (DMAc), N-methyl-2-pyrrolidone, γ-butyrolactone, diethyl ether, ethylene glycol dimethyl ether, diglyme, tetrahydrofuran, acetylacetone, acetonitrile, chloroform, dichloromethane, tetrachloroethane, trichloroethylene, tetrachloroethylene, chlorobenzene, and benzene.

When a mixture of solvents is used, the ratio of the solvents is generally not critical and may vary from 99:1 to 1:99 weight-to-weight, provided that the solvent mixture is able to dissolve the MOF components. It will be appreciated by those skilled in the art that the concentration of the MOF components in the organic solvent may be adjusted by removing a portion of the organic solvent or by adding more of the organic solvent, as may be desired.

While not wishing to be bound by any theory, it is understood that addition of the electron-donating encapsulant to the electron-deficient MOF results in formation of the donor-acceptor complex inside the OF structure, wherein the electron-donating encapsulant is complexed to the electron-deficient ligand of the metal-ligand complex by way of a charge transfer. This interaction (often called a host-guest interaction) leads to enhanced fluorescence of the weakly emissive electron-deficient MOF. The resulting donor-acceptor complexes may therefore serve as a luminescent and colorimetric probe for the detection of strongly basic organic amines such as fentanyl and its derivatives. This host-guest binding phenomena was described in the article of Liu J. J. et al. “Encapsulating Naphthalene in an Electron-Deficient MOF to Enhance Fluorescence for Organic Amines Sensing” (Inorg. Chem. 2016, 55, 3680) which is incorporated herein in its entirety by reference.

The present embodiments of the invention further provide a composition for detecting organic amines. The composition includes a solid support impregnated with an indicator reagent including a metal-organic framework structure. The description of the solid support and the indicator reagent including the metal-organic framework structure is the same as above and will not be repeated here.

The present embodiments of the invention further provide a method of detecting organic amines by colorimetric visualization using the indicator reagent including the metal-organic framework structure. The method includes exposing an article comprising a solid support impregnated with an indicator reagent to a medium including an organic amine to produce a color change of the indicator reagent. The method may farther include visually detecting the color change indicating the presence of the organic amine in the medium. The description of the solid support and the indicator reagent including the metal-organic framework structure is the same as above and not be repeated here.

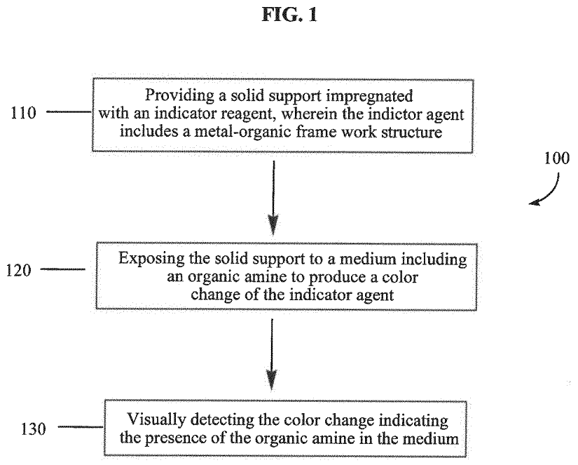

An embodiment of the method is illustrated FIG. 1 at 100. The operations of method 100 are intended to be illustrative, not limiting. Method 100 may be accomplished with one or more additional operations not described, and/or without one or more of the operations. Additionally, the order in which the operations of method 100 is performed and described below is not intended to be limiting. As shown in FIG. 1 in operation 110 a solid support may be provided. The solid support may be impregnated with an indicator reagent as described herein, wherein the indicator reagent includes a metal-organic framework structure. In operation 120, the solid support is exposed to a medium including an organic amine as described herein, to produce a color change of the indicator reagent. In operation 130, the color change of the indicator reagent is visually detected thereby indicating the presence of the organic amine in the medium.

Visual detection can occur by a multitude of techniques, including, among others, colorimetric, fluorescence, or chemiluminescence analysis. Colorimetric analysis has been widely used for qualitative and quantitative determination of a chemical element or compound in a solution with a help of an indicator reagent. Color originates from the interaction of the visible light with chromophores, or groups of atoms in molecules, which are capable of absorbing certain wavelengths of visible light. Due to this absorption phenomenon, these substances tire perceived as having color. The hue and depth of the perceived color are related to the specific wavelengths absorbed and the intrinsic absorption efficiency of the molecule, respectively. The distribution of electron density in a molecule is responsible for the absorption characteristics of that molecule. The alteration of electron density distribution in a molecule through reaction with another chemical species forms the basis for colorimetric chemical detection.

The article including a solid support impregnated with an indicator reagent including a metal-organic framework structure is useful for detection of organic amines. When the solid support containing the metal-organic framework structure is exposed to the organic amine, the organic amine coordinates to the metal-organic framework, resulting in a color change. The color change indicating the presence of amine may he observed visually or detected with a help of an appropriate instrument.

The organic amine may have a tertiary amine structure. The tertiary amine structure is common to many potent opioid drugs, such as fentanyl and its derivatives. Fentanyl (ED₅₀ is 0.08 mg/kg) is a potent synthetic opioid that is 50-100 times stronger than morphine. Pharmaceutical fentanyl was developed for pain management treatment of cancer patients, applied in a patch on the skin. However, due to its powerful opioid properties, fentanyl often becomes diverted for abuse. Fentanyl is added to heroin to increase its potency, or be disguised as highly potent heroin. Many drug users believe that they are purchasing heroin and actually do not know that they are purchasing fentanyl. On the other hand, even traces of fentanyl dramatically change potency of the fentanyl-laced heroin. The use of a lethal fentanyl dose often therefore results in overdose deaths.

Derivatives of fentanyl are highly potent substances with low lethal doses, which may be even more potent than fentanyl. Among these derivatives are mefentanyl (HD₅₀ is 0.0001 mg/kg), phenaridine (ED₅₀ is 0.0048 mg/kg), α-mefentanyl, ohmefentanyl (ED₅₀ is 0.0001 mg/kg),, carfentanyl (ED₅₀ is 0.00032. mg/kg), lofentanyl (ED₅₀ is 0.2 mg/kg), sufentanyl, alfentanil (ED₅₀ is 0.044 mg/kg), brifentanil (ED₅₀ is 0.047 mg/kg), remifentanil (ED₅₀ is 0.0044 mg/kg), trefentanyl, and mirfentanil (ED₅₀ is 0.07 mg/kg):

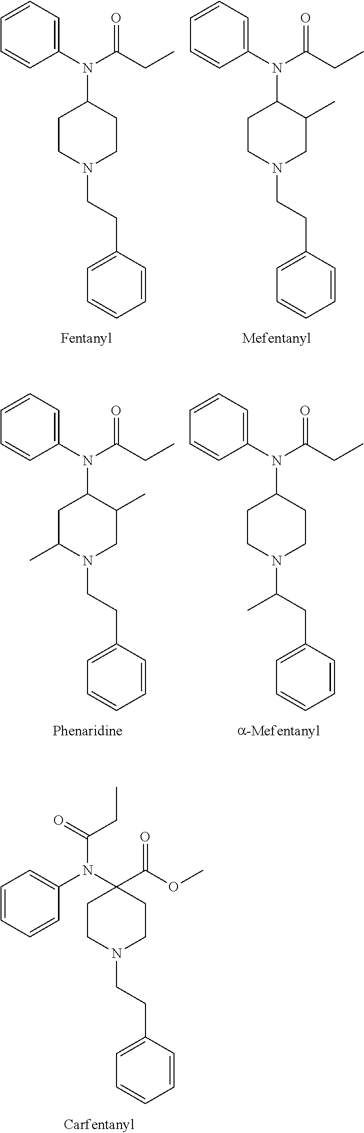

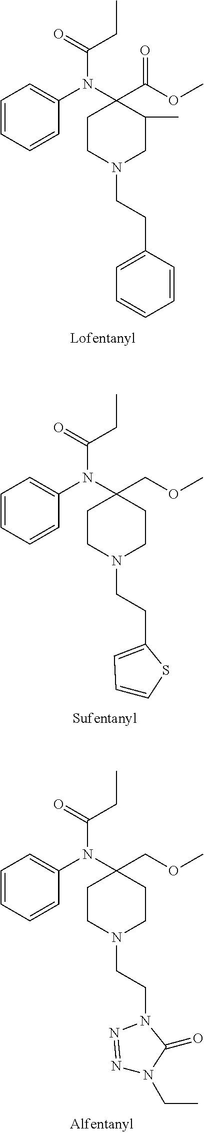 [SMILES: CCC(=O)N(c1ccccc1)C1(C(=O)OC)CCN(CCc2ccccc2)CC1C ; CCC(=O)N(c1ccccc1)C1(COC)CCN(CCc2cccs2)CC1 ; CCC(=O)N(c1ccccc1)C1(COC)CCN(CCn2nnn(CC)c2=O)CC1]

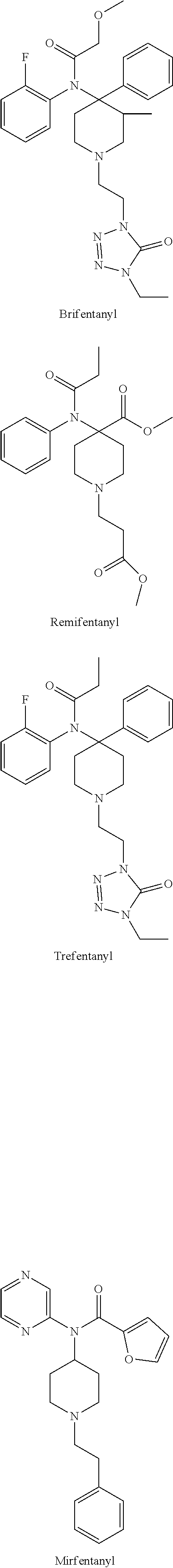 [SMILES: CCn1nnn(CCN2CCC(c3ccccc3)(N(C(=O)COC)c3ccccc3F)C(C)C2)c1=O ; CCC(=O)N(c1ccccc1)C1(C(=O)OC)CCN(CCC(=O)OC)CC1 ; CCC(=O)N(c1ccccc1F)C1(c2ccccc2)CCN(CCn2nnn(CC)c2=O)CC1 ; O=C(c1ccco1)N(c1cnccn1)C1CCN(CCc2ccccc2)CC1]

Because of the high sensitivity of the MOF, the present method allows to visually detect fentanyl and its derivatives by the presently described method. Specifically, upon exposure, MOP will display distinct colorimetric response to even low concentrations of the fentanyl and its derivatives which may be detected by using the article of the present embodiments of the invention.

Embodiments of the present invention further provide devices including an article for detecting organic amines. The disclosed article may be incorporated in various devices to facilitate detection of the organic amines. Such devices and principles of their operation have been disclosed in U.S. Pat. No. 5,648,047, U.S. Pat. No. 6,895,889, U.S. Pat. No. 8,980,641, and U.S. Pat. No. 9,880,092, the contents of which are incorporated herein in their entireties by reference.

The article for colorimetric detection of organic amines according to the present embodiments of the invention has the following advantages. The article is hand-held, does not require a power source for its operation, and can be used for rapid and repetitive colorimetric detection opioid drugs. Therefore, it can be easily and accurately operated by non-skilled personnel. The article can be utilized without exposing the user to hazardous reagents and without exposing sensitive reagents to deteriorating environmental conditions.

The present inventive concept is further illustrated by the following examples. All compounds and reagents used herein are available commercially except where a procedure is provided below.

### EXAMPLES

### Synthesis Of Metal-Organic Frameworks

N,N′-Di-(4-pyridyl)-1,4,5,8-naphthalenetetracarboxydiimide (DPNDI)

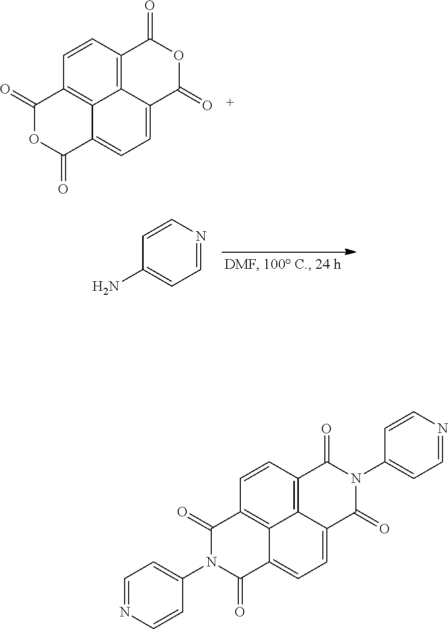 [SMILES: O=C1OC(=O)c2ccc3c4c(ccc1c24)C(=O)OC3=O ; Nc1ccncc1 ; O=C1c2ccc3c4c(ccc(c24)C(=O)N1c1ccncc1)C(=O)N(c1ccncc1)C3=O]

1,4,5,8-naphthalenetetracarboxylic dianhydride (0.805 g, 3.00 mmol) and 4-aminopyridine (0.847 g, 9.01 mmol) were dissolved in dimethyl acetamide (150 mL) and heated to 135° C. under Ar for 18 h. The reaction mixture was cooled to room temperature and poured into diethyl ether (600 mL). The resulting precipitate was filtered and washed with diethyl ether. The product was dried under vacuum to give DPNDI (1.07 g, 2.54 mmol, 85%). (400 MHz, DMSO w/1% v/v CF₃COOH) δ 9.09 (br s, Hz, 4 H), 8.78 (br s, 4 H), 8.07 (br s, 4 H), as described in Crystal Growth and Design 2009, 9(7), 3327-3332, which is incorporated herein in its entirety by reference.

ZnSiF₆(DPNDI)₂

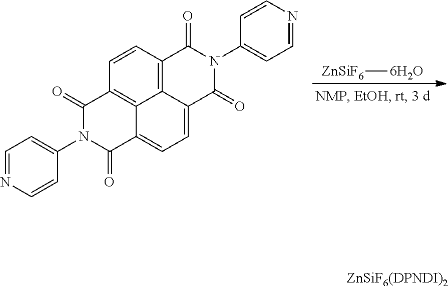

A 250 mL round bottom flask containing a solution of DPNDI (0.200 g, 0.475 mmol) in NMP (50 mL) was carefully layered with a solution of ZnSiF₆·6H₂O (0.30( )g, 0.951 mmol) in EtOH (40 mL) and NMP (10 mL), After several days in the dark, a pale yellow precipitate was observed. The product was filtered, washed with mother liquid and then immersed in ethanol to stand for 3 d. The resulting solids were filtered and dried in vacuum to give ZnSiF₆(DPNDI)₂ as a pale yellow solid.

### ZnSiF₆(DPNDI)2⊃Naphthalene

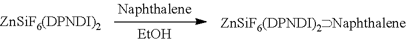

ZnSiF₆(DPNDI)₂ (100 mg) was immersed in a 300 mM solution of naphthalene in ethanol (20 mL). The mixture was allowed to stand for 3 d. The resulting solids were filtered and washed with ethanol several times and the resulting solids were dried in vacuum to give ZnSiF₆(DPNI)₂⊃Naphthalene as an orange solid.

ZnSiF₆(DPNDI)₂⊃Indole

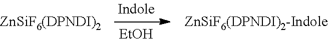

ZnSiF₆(DPNDI)₂⊃Indole was prepared according to the procedure for ZnSiF₆(DPNDI)2⊃Naphthalene, except that indole was used as an encapsulant instead of naphthalene.

ZnSiF₆(DPNDI)₂⊃Quinoline

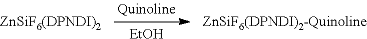

ZnSiF₆(DPNDI)₂⊃Quinoline was prepared according to the procedure for ZnSiF₆(DPNDI)₂⊃Naphthalene, except that indole was used as an encapsulant instead of naphthalene.

CuSO₄(DPNDI)₂

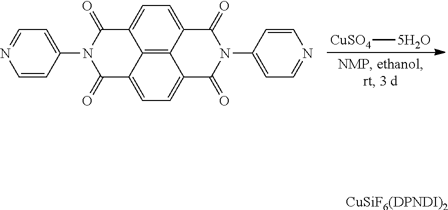

A 250 mL round bottom flask containing a solution of DPNDI (0.150 g, 0.357 mmol) in NMP (60 mL) and ethanol (10 mL) was treated with CuSO₄·5H₂O (0.178 g, 0.714 mmol). After several days in the dark, a pale yellow green precipitate was observed. The product was filtered, washed with mother liquid and then immersed in ethanol to stand for 3 d. The resulting solids were filtered and dried in vacuum to give CuSO₄(DPNDI)₂, as a light yellow/ green solid.

CuSO₄(DPNDI)₂⊃Naphthalene

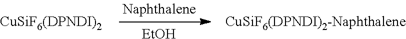

CuSO₄(DPNDI)₂ (100 mg) was immersed in a 300 mM solution of naphthalene in ethanol (20 mL). The mixture was allowed to stand for 3 d. The resulting solids were filtered and washed with EtOH several times and the resulting solids were dried in vacuum to give CuSO₄(DPNDI)₂⊃Naphthalene as a green solid.

### Colorimetric Detection of Organic Amines

Filter paper was functionalized with the encapsulated MOF by pressing into Whatman filter paper (Grade 1) to deposit the solid residue and the excess material. The filter paper was cut into 1 cm wide strips or 1 cm circles for testing.

General testing procedure. A solution of fentanyl in ethanol (20-200 μL) was deposited onto the MOF-impregnated paper and allowed to dry over 30 min. Color change was compared to a control sample treated with the same amount of alcohol solvent, as shown in FIG. 2.

ZnSiF₆(DPNDI)₂⊃Naphthalene dried on filter paper was treated with fentanyl citrate. The colorimetric response is shown in FIG. 2.

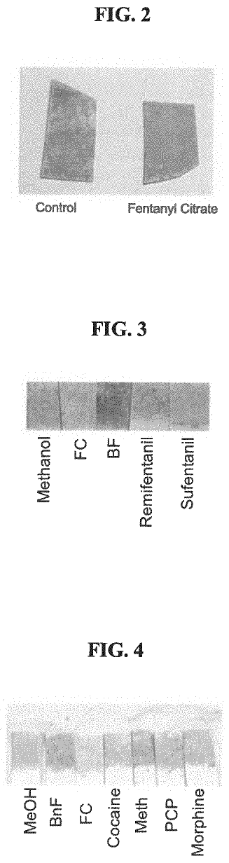

FIG. 3 shows colorimetric response of fentanyl-sensing metal-organic frameworks (FMOF) with respect to some drug substances. From left to right: methanol (control), fentanyl citrate (FC), benzyl fentanyl oxalate (BF), remifentanil hydrochloride, sufentanil hydrochloride using 200 μL of a 10 mg/mL stock solution.

FIG. 4 shows colorimetric response of FMOF with respect to some other drug substances. From left to right: methanol (control), benzyl fentanyl (BF), fentanyl citrate (FC), cocaine, methamphetamine, PCP, morphine. This test was performed using 100 μof 25 mg/mL stock solutions.

The present inventive concept has been described in terms of exemplary principles and embodiments, but those skilled in the art will recognize that variations may be made and equivalents substituted for what is described without departing from the scope and spirit of the disclosure as defined by the following claims.

What is claimed is:

1. An article for detecting organic amines, comprising a solid support impregnated with an indicator reagent comprising a meal-organic framework structure.

2. The article according to claim 1, wherein the metal-organic framework structure comprises a plurality of metal ions and an electron-deficient bidentate ligands coordinated to the metal ions.

3. The article according to claim 2, wherein the metals ions are transition metal ions,

4. The article according to claim 3, wherein the transition metal ions are independently selected from Cu, Zn²⁺, Zr²⁺, Mi²⁺, and Co²⁺.

5. The article according to claim 2, wherein the electron-deficient bidentate ligand is a dicarboxylic acid ligand or a heteroaromatic ligand comprising at least two nitrogen atoms.

6. The article according to claim 5, wherein the dicarboxylic acid ligand is represented by Formula 3:
HOOC—L¹—COOH Formula 3
wherein, in Formula 1, L¹ is a moiety comprising a substituted or unsubstituted C2-C30 alkenylene group, a substituted or unsubstituted C2-C30 alkynylene group, a substituted or unsubstituted C3-C30 cycloalkenylene group, a substituted or unsubstituted C3-C30 cycloalkynylene group, a substituted or unsubstituted C6-C30 arylene group, or a substituted or unsubstituted C6-C30 heteroarylene group, a C1-C30 alkylene group, wherein at least one non-adjacent —CH₂- group is replaced by —SO₂—, —C(═O)—, —O—, —S—, —SO—, —C(═O)O-, or —C(═O)NR-, wherein R is hydrogen or a C1-C10 alkyl group, or a combination thereof.

7. The article according to claim 5, wherein the heteroaromatic ligand comprising at least two nitrogen atoms is represented by Formula 4:
N-RING—L₂—N-RING Formula 4
wherein, in Formula 4,
N-RING is the same or different and is a nitrogen-containing heterocycle; and
L² is a single bond or a moiety comprising a substituted or unsubstituted C2-C30 alkenylene group, a substituted or unsubstituted C2-C30 alkynylene group, a substituted or unsubstituted C3-C30 cycloalkenylene group, a substituted or unsubstituted C3-C30 cycloalkynylene group, a substituted or unsubstituted C6-C30 arylene group, or a substituted or unsubstituted C6-C30 heteroarylene group, a C1-C30 alkylene group, wherein at least one non-adjacent —CH₂- group is replaced by —SO₂—, —C(═O, —O—, —S—, —SO—, —C(═O)O-, or —C(═O)NR-, wherein R is hydrogen or a C1-C10 alkyl group, or a combination thereof.

8. The article according to claim 7, wherein the heteroaromatic ligand comprising at least two nitrogen atoms is represented by Formula 5: 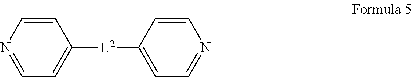
wherein, in Formula 2, L² is a single bond or a moiety comprising a substituted or unsubstituted C2-C30 alkenylene group, a substituted or unsubstituted C2-C30 alkynylene group, a substituted or unsubstituted C3-C30 cycloalkenylene group, a substituted or unsubstituted C3-C30 cycloalkynylene group, a substituted or unsubstituted C6-C30 arylene group, or a substituted or unsubstituted C6-C30 heteroarylene group, a C1-C30 alkylene group, wherein at least one non-adjacent —CH₂- group is replaced by —SO₂—, —C(═O)—, —O, —S—, —SO, —C(═)O-, or —C(═O)NR-, wherein R is hydrogen or a C1-C10 alkyl group, or a combination thereof.

9. The article according to claim 6, wherein the dicarboxylic acid ligand is represented by one of the following formulae:

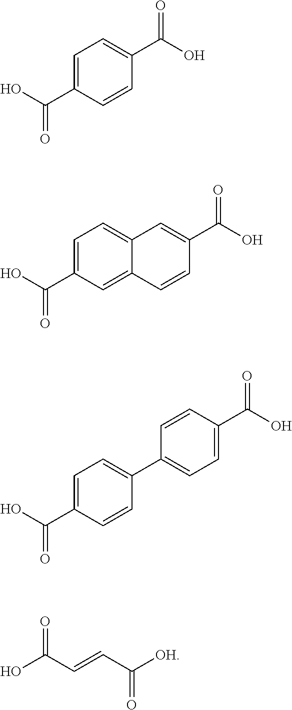 [SMILES: O=C(O)c1ccc(C(=O)O)cc1 ; O=C(O)c1ccc2cc(C(=O)O)ccc2c1 ; O=C(O)c1ccc(-c2ccc(C(=O)O)cc2)cc1 ; O=C(O)/C=C/C(=O)O]

10. The article according to claim 8, wherein the heteroaromatic ligand comprising at least two nitrogen atoms is represented by one of the following formulae:

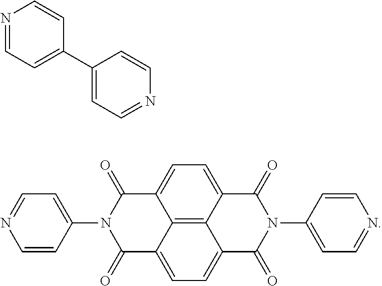 [SMILES: c1cc(-c2ccncc2)ccn1 ; O=C1c2ccc3c4c(ccc(c24)C(=O)N1c1ccncc1)C(=O)N(c1ccncc1)C3=O]

11. The article according to claim 1, wherein the solid support is polymeric fiber.

12. The article according to claim 11, wherein the polymeric fiber is porous paper, a porous polymer, or cotton.

13. The article according to claim 2, wherein the metal-organic framework structure further comprises an encapsulant.

14. The article according to claim 13, wherein the encapsulant is at least one selected from a monocyclic aromatic compound, a bicyclic aromatic compound, a monocyclic heteroaromatic compound, or a bicyclic heteroaromatic compound.

15. The article according to claim 13, wherein the encapsulant forms a donor-acceptor complex with the electron-deficient ligand.

16. The article according to claim 1, wherein the organic amine is: a substituted or unsubstituted N-phenyl-N-[1-(2-phenylethyl)piperidin-4-yl]propenamide (fentanyl),
a substituted or unsubstituted N-(3-methyl-1-phenethylpiperidin-4-yl)-N-phenylpropionamide (mefentanyl),
a substituted or unsubstituted N-(2,5-dimethyl-1-phenethyipiperidin-4-yl)-N-phenylpropionamide (phenaridine),
a substituted or unsubstituted N-phenyl-N-(1-(1-phenylpropan-2-yl)piperidin-4-yl)propionatnide (α-mefentanyl),
a substituted or unsubstituted methyl 1-phenethyl-4-(N-phenylpropionamido)piperidine-4-carboxylate (carfentanyl),
a substituted or unsubstituted methyl 3-methyl-1-phenethyl-4-(N-phenylpropionamido)piperidine-4-carboxylate (lofentanyl),
a substituted or unsubstituted N-(4-(methoxymethyl)-1-(2-(thiopben-2-yl)ethyl)piperidin-4-yl)-N-phenylpropionamide (sufentanyl),
a substituted or unsubstituted N-(1-(2-(4-ethyl-5-oxo-4,5-dihydro-1H-tetrazol-1-yl)ethyl)-4-(methoxymethyl)piperidin-4-yl)-N-phenylpropionamide (alfentanyl),
a substituted or unsubstituted N-(1-(2-(4-ethyl-5-oxo-4,5-dihydro-1H-tetrazol-1-yl)ethyl)-3-methyl-4-phenylpiperidin-4-yl)-N-(2-fluorophenyl)- 2-methoxyacetamide (brifentanil),
a substituted or unsubstituted methyl 1-(3-methoxy-3-oxopropyl)-4-(N-phenylpropionamido)piperidine-4-carboxylate (remifentanil),
a substituted or unsubstituted N-(1-(2-(4-ethyl-5-oxo-4,5-dihydro-1H-tetrazol-1-yl)ethyl)-4-phenylpiperidin-4-yl)-N-(2-fluorophenyl)propionamide (trefentanyl), or
a substituted or unsubstituted N-(1-phenethylpiperidin-4-yl)-N-(pyrazin-2-yl)furan-2-carboxamide (mirfentanil)

17. A device comprising the article according to claim 1.

18. A composition comprising a solid support impregnated with an indicator reagent, wherein the indicator reagent comprises a metal-organic framework structure.

19. A method of detecting organic amines, comprising:
exposing an article comprising a solid support impregnated with an indicator reagent to a medium including an organic amine to produce a color change of the indicator reagent, wherein the indicator reagent comprises a metal-organic framework structure.

20. The method according to claim 19, further comprising:
detecting the color change, wherein the color change indicates a presence of the organic amine in the medium.
